(19) United States
(12) Patent Application Publication
(10) Pub. No.: US 2004/0228897 A1
(43) Pub. Date: Nov. 18, 2004

(54) Methods and apparatus for in vivo cell localization

(76) Inventors: Ping Ye Zhang, San Diego, CA (US); Yi Zhang, San Diego, CA (US); Xiu-Ming Yang, San Diego, CA (US)

Correspondence Address: CATALYST LAW GROUP, APC, 4330 LA JOLLA VILLAGE DRIVE SUITE 220, SAN DIEGO, CA, 92122, US

(21) Appl. No.: 10/439,495

(22) Filed: May 16, 2003

(51) Int. Cl. A61F 2/00

(52) U.S. Cl. 424/423; 424/93.7

(57) ABSTRACT

The present invention includes a medical device for use to assist stem cell and/or stem cell derivatives in repopulating, repairing and/or replacing the heart tissue in a failing heart muscle, in order to restore the heart's ability to pump blood. The medical device is made of biocompatible materials. The specific design of the device will facilitate the stem cells coated in the device to repopulate heart muscles inside the heart. Stem cells are attached to the coated device, proliferated and/or differentiated on the device in a bioreactor before implantation. The device also contains bioactive components that diminish rejection by the host's immune system. The device may be directly implanted into the failing heart muscle area to assist stem cells to repair failing heart muscles via surgical and/or percutaneous catheter based procedures. In another embodiment, the device may be implanted to the surgical site where abnormal heart muscles are removed, to assist stem cells to repopulate heart muscles, to replace the failing heart muscles.

### FIELD OF THE INVENTION

The present invention relates to the field of methods and apparatus to transplant stem cell and cardiomyocyte derivatives into failing heart muscle to cure heart disease. In particular, the present invention relates to a medical device for use to assist stem cells in repopulating the heart tissue, and repairing and/or replacing the failing heart muscle, in order to restore the heart's ability to pump blood.

### DESCRIPTION OF RELATED ART

When heart muscle is damaged by injury such as a heart attack, functional contracting heart muscle dies and is replaced with nonfunctional scar tissues. Heart attacks cause massive loss of heart muscle cells, known as cardiomyocytes, resulting in a diminished heart pumping ability. This year, it is estimated that 1.1 million people will have a heart attack, which is the primary cause of heart muscle damage. Heart muscle can also be damaged by coronary artery disease. Coronary artery disease leads to the episodes of cardiac ischemia, in which the heart muscle is not getting enough oxygen-rich blood. Eventually, the heart muscle enlarges from the additional work it must do in the absence of enough oxygen-rich blood, leading to ischemic cardiomyopathy—a type of heart disease in which the heart is abnormally enlarged, thickened and/or stiffened. As a result, the heart muscle's ability to pump blood is usually weakened. This damage commonly results in congestive hear failure. Congestive heart failure affects more than four million people in the United States. Although heart transplantation has proven very successful in the treatment of heart failure, only a small number of organs are actually available for transplant.

In general, stem cells are naturally occurring, self-renewing and undifferentiated primitive cells that develop into any of a number of functional, differentiated cells. For example, human embryonic stem cells are pluripotent: that is, they can develop into all cells and tissues in the body and thereby perform a specialized function. (Bishop, A., et al 2002. Embryonic stem cells. J Pathol 2002: 197: 424-429.) There are various types of human stem cells, also called hSCs: (1) human embryonic stem cells (hES), which are derived from donated in vitro fertilized blastocysts or very early-stage embryos; (2) human embryonic gem cells (hEG), which are derived from donated fetal material; (3) human embryonic carcinomas cells (hEC) derived from embryonal carcinomas; and (4) adult stem cells (hS), which are derived from tissues such as bone marrow, spleen or blood cells. Cardiomyocytes derived from stem cells are suggested for use with cellular transplantation therapy in humans suffering from congestive heart failure and the heart muscle damage caused by heart attack. (Penn, S. M., et al 2002. Autologous cell transplantation for the treatment of damaged myocardium. Progress in Cardiovascular disease. 45: 21-32; Grounds, M. D., et al 2002. The role of stem cells in skeletal and cardiac muscle repair. J. H. C. 50: 589-610; Kehat, I., et al 2001. Human embryonic stem cells can differentiate into myocytes with structural and functional properties of cardiomyocytes. J. Clin. Invest. 108: 407-414; Jackson, K. A., et al 2001. Regeneration of ischemic cardiac muscle and vascular endothelium by adult stem cells. J. Clin. Invest. 107, 1-8; Hagege, A. A., et al 2001. Myoblast transportation for heart failure. Lancet. 357: 279-280; Hescheler, J., et al 2001. Indispensable tools: embryonic stem cells yield insights into the human heart. J. Clin. Invest. 108: 363-364.)

Researchers have demonstrated this concept in mice, using mouse cardiomyocytes derived from mouse embryonic stem cells. When injected into the hearts of recipient adult mice, the cardiomyocytes repopulate the heart tissue and stably integrate into the muscle tissue of the adult mouse heart. (Stamm, C., et al 2003. Autologous bone-marrow stem cell transplantation for myocardial regeneration. Lancet. 361: 45-46; U.S. Pat. No.: 6,534,052B1; and U.S. Pat. No.: 6,387,369.)

Stem cell transplants have since been used to regenerate cell populations in humans. For instance, in June of 2000 a 72 year old man suffering from chronic heart failure due to a past heart attack underwent the procedure. Stem cells transplanted in the patient's heart muscle tissue were able to replace the cardiac muscle that had been damaged by the prior heart attack. (Hagege, A. A., et al 2003. Viability and differentiation of autologous skeletal myoblast graft in ischemic cardiomyopathy. Lancet. 361: 491-492.)

Similar success has been shown using stem cells harvested from sources such as bone marrow and blood. In April 2002, Australian surgeons carried out a trial using bone-marrow stem cells to repair heart damage in a 74-year-old man. Earlier 2003, German and Hong Kong researchers conducted a similar procedure in heart attack patients.

For the above mentioned animal and clinical studies, a bolus of stem cells has been injected locally to the desired sites. Generally the bolus is a cell suspension in an appropriate medium, and may include a polymerization solution. Whether the cells are delivered in medium alone or with a polymerization agent, an optimum distribution and survival of initial stem cells deposits are not achieved. Stem cells injected on the surface of the heart muscle naturally migrate away from the injection site followed by apoptosis. Additionally, the transplanted stem cells that do take hold at the area of interest will not reach the necessary depth of the failing heart muscle to sufficiently repopulate the heart muscle and regenerate its pumping function.

U.S. Pat. No.: 6,537,567 B1 titled Tissue-Engineered Tubular Construct Having Circumferentially Orientated Smooth Muscle Cells is directed towards ex vivo blood vessel tissue generation using constructs to facilitate cell growth. These constructs are specific-defined tubular structures with internal lumen that withstands an internal shear force, and therefore this structure is more applicable for replacement of a functional vessel, and is not suitable for large surface replacement of damaged tissues.

Thus, there is a need in the art to develop methods and apparatus which are useful for delivering a sufficiently confluent population of cells to a target area, said delivery providing optimum breadth and depth of cell repopulation in a target tissue and/or organ thus facilitating the in vivo repair and/or replacement of said tissue and/or organ in a tissue or an organ specific manner. Ideally, the delivery method will also be minimally invasive to the host. Such improvements in implanting and localizing stem-cell transplants will prove beneficial to numerous transplant and grafting procedures, including, but not limited to stem cell transplant in the heart to repair and/or replace failing heart muscle, whether during surgical or via percutaneous catheter-based implantation.

### SUMMARY OF THE INVENTION

The present invention is directed to a method and apparatus for transplanting stem cells into a specific area, such as the heart. In one embodiment, the present invention system includes a cell growth matrix, such as a meshed tube or a group of meshed tubes placed into an area of damaged heart muscle; one or both sides of said tubes being coated with a controlled amount of stem cells for distribution to the heart (generally referred to as a cell coated device or meshed tube); a means of depositing stem cells onto the tubes and means of attaching the tubes to the heart muscle. The mesh design has specific patterns to facilitate stem cells growth direction. Materials for constructing said tubes are well known in the art and include metal materials such as stainless steel, nitinol or are made of bioabsorbable or/and biocompatible materials, as are generally recited in U.S. Pat. No.: 6,537,567. Cells are adhered to the matrix and are grown to a desired confluence or concentration under appropriate conditions. The cell coated matrix is then placed in a target location within the body, where the cells will properly adapt and grow compatibly with the organ's or tissue's individualized growth pattern. Placement of the cell coated matrix/device into a tissue or organ is done via surgery or percutaneous delivery catheter. Because the cells are adhered to the cell coated device, there is not a problem with loss of cell concentration due to cells drifting away from the location. Additionally, because the cell coated device can be inserted into a tissue or organ, proper depth of the cells is readily achieved, allowing the cells to fully repopulate the damaged organ. Thus, both the appropriate depth into the tissue or organ and the desired cell concentration are readily achieved. As used with damaged heart muscle, the in-depth localized stem cell distribution approach in an area of damaged heart muscle will assist stem cells to sufficiently repopulate the heart muscle, and in turn will restore the heart pumping function.

In an alternative embodiment, the present invention includes a pad or a series of pads inserted into an area of damaged heart muscle; one side or both sides of the pads are coated with controlled amount of stem cells to be distributed in the heart (generally referred to as a cell coated device or pad); and a means for attaching the pads to the heart muscle. The pads may be made of metal materials such as stainless steel, nitinol or of bioabsorbable or/and biocompatible materials.

In another embodiment, if heart muscle damage occurs on the surface of the heart, the present invention includes a pad and means for attaching said pad to the surface area of damaged heart muscle.

In other embodiments, the in-depth stem cell transplantation approach and surface stem cell transplantation approach may be used as combination.

In other embodiments, after the abnormal heart muscle is surgically removed, i.e., in the case of cardiomyopathy—a type of heart disease in which the heart is abnormally enlarged, thickened and/or stiffened—the stem-cell coated matrices are implanted into the surgical site via the device of the current invention, to repopulate heart muscles needed for pumping function.

### BRIEF DESCRIPTION OF THE DRAWINGS

The present invention is best understood by referring to the following description and accompanying drawings that are used to illustrate embodiments of the invention. In the drawings:

FIG. 1 is a perspective view of one embodiment of the present invention in use to assist stem cell repopulation of the heart tissue in the failing heart muscle area.

FIG. 2 is a perspective view of the embodiment of FIG. 1 coated with stem cell formula.

FIG. 3 is a top view of the embodiment of FIG. 1 implanted into failing heart muscle area.

FIG. 4 is a cross section view and a transverse plane view of the embodiment of FIG. 1 implanted into failing heart muscle area

FIG. 5 is a perspective view of an alternative embodiment of the present invention used to assist stem cell to repopulate the heart tissue in the failing heart muscle area.

FIG. 6 is a top view of the embodiment of FIG. 5 implanted into failing heart muscle area.

FIG. 7 is a cross section view and a transverse plane view of the embodiment of FIG. 5 implanted into failing heart muscle area.

FIG. 8 is a perspective view of the embodiment of FIG. 5 attached to the surface of the failing heart muscle area.

FIG. 9 is a top view of the embodiment of FIG. 1 placed into the failing heart muscle area and the embodiment of FIG. 5 attached to the surface of such area.

FIG. 10 is a top view of the embodiment of FIG. 5 placed into the failing heart muscle area and the embodiment of FIG. 5 attached to the surface of such area.

FIG. 11 is a perspective view of the embodiment of FIG. 1 implanted to surgical site where abnormal heart muscles such as failing; enlarged, stiffened heart muscles are surgically removed.

FIG. 12 is a perspective view of the embodiment of FIG. 5 implanted to surgical site where abnormal heart muscles such as failing; enlarged, stiffened heart muscles are surgically removed.

FIG. 13 is a perspective view of the catheter delivery system used in the present invention implantation per percutaneous catheter approach.

FIG. 14 is a perspective view of the present invention delivered via percutaneous catheter to failing heart muscle area.

### DETAILED DESCRIPTION OF THE PREFERRED EMBODIMENT

The present invention provides a method and apparatus that helps transplant stem cells or differentiated cells into a heart to repair and/or replace failing heart muscle. While the present invention is described in detail as applied to failing heart muscle repair and/or replacement, those of ordinary skill in the art will appreciate that the present invention can be applied to other organs.

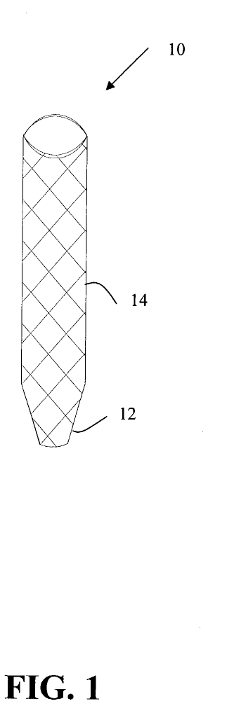

FIG. 1 illustrates a general example of meshed hollow tube according to one embodiment of the present invention. In one embodiment, the meshed tube 10 includes a point tip 12 and main body 14. Use of point tip 12 allows the meshed hollow tube 10 to be easily inserted into the failing heart muscle to reach to desired depth. The meshed tube 10 forms the scaffolding matrix upon which stem cells are seeded and grown for transplantation. Preferably, the scaffolding matrix is a biocompatible material and more preferably a biodegradable or bioerodable.

In a preferred embodiment of the present invention illustrated of FIG. 1, the meshed tube 10 may be fabricated from 304V stainless steel. Alternatively, other biocompatible metal materials such as nitinol may be used. In one exemplary embodiment, the present invention may be fabricated from bioabsorbable materials such as poly lactic acid (PLA), polyglycolic acid (PGA), polysebacic acid (PSA), poly(lactic-co-glycolic) acid copolymer (PLGA),poly(lactic-co-sebacic) acid copolymer (PLSA), poly(glycolic-co-sebacic) acid copolymer (PGSA), polyesters, polyorthoesters, polyanhydrides, polyiminocarbonates, inorganic calcium phosphate, aliphatic polycarbonates, polyphosphazenes, collagen based adhesive, fibrin based adhesive, albumin based adhesive, polymers or copolymers of caprolactones, amides, amino acids, acetals, cyanoacrylates, degradable urethanes; or biocompatible but non-bioabsorable materials such as acrylates, ethylene-vinyl acetates, non-degradable urethanes, styrenes, vinyl chlorides, vinyl fluorides, TEFLON® (DuPont, Wilmington, Del.), nylon, HYTREL (DuPont) or PEBAX (Autofina). The above disclosure is not an exhaustive list, but instead represents alternate embodiments illustrated by way of example only. Those of ordinary skill in the art are knowledgeable of and will readily employ the numerous biocompatible, biodegradable and bioerodable materials in the art in order to achieve the spirit of the current invention.

Both the point tip 12 and the main tube body 14 are coated with stem cell formula 16. Stem cell formula 16 is generally a medium well known in the art that will maintain stem cells allowing their adherence to meshed tube 10 and assuring their viability during the transplant procedure. Those of ordinary skill in the art will readily employ a variety of cell culture techniques to the current invention to achieve substantially similar results.

Although the embodiment of the present invention illustrated in FIG. 1 shows the device 10 is a meshed uniform tube along the length except for the point tip 12, and is hollow though both ends, it is understood by those of ordinary skill in the art that other embodiments in which device may be formed by different configurations. For example, other embodiments may include, but are not limited to solid cylindrical spikes, knife like blades, large surface area patterns such as spike covered spheres, or custom shapes dictated by the shape of the tissue or organ area to be replaced. In further embodiments, the device 10 may have an alternate shaped cross section, i.e. square, rectangle, diamond, etc., and the cross section area may be varied along the length of the device, or, there may or may not be point tip 12. In an alternative embodiment, one end or both ends of device 10 may be closed or pattern cut and coated with stem cell formula 16.

Although the embodiment of the present invention illustrated in FIG. 1 may be fabricated by pattern cutting a tube, other approaches to fabricate the meshed tube may be used. In one exemplary embodiment, a meshed pad with stem cell coated may be folded to form a tube. Hooks at the ends of the pad may be created to secure the folding.

Although the embodiment of the present invention illustrated in FIG. 1 may have meshed design cuts. Other pattern designs may be used. In one exemplary embodiment, the present invention may have a surface or surfaces with cuts parallel to the device 10 axis. The purpose of specific pattern design is to facilitate the stem cell's growth depth and orientation inside the failing heart muscle area, to restore the heart muscle pumping function.

In one embodiment, device 10 may have specific cut pattern design going through the wall thickness. It is to be understood that device 10 may have specific cut pattern design(s) on both sides, but the cuts may not go through the wall thickness.

Alternatively, in one embodiment, device 10 may not have patterned cuts. In this instance, device 10 may have smooth or rough surface(s) on one side or both sides.

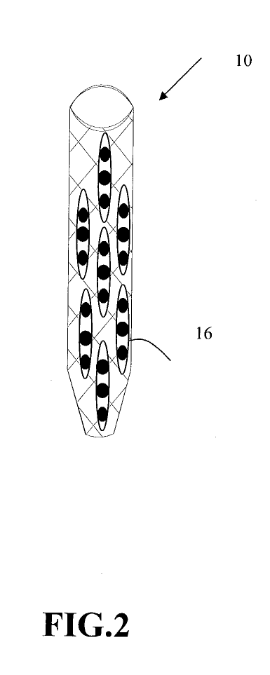

FIG. 2 illustrates partial perspective view of the embodiment of FIG. 1 where stem cell formula 16 is coated onto device 10, thus resulting in a cell coated device. Stem cell formula 16 may be coated onto either exterior surface 20 or interior surface 22, or both sides. Stem cell formula 16 is generally a medium well known in the art that will maintain undifferentiated stem cells allowing their adherence to meshed tube 10 and assuring their viability during the transplant procedure.

Sources of stem cells on the meshed tubes 10 could be from human embryonic stem cells (hES), human embryonic germ (hEG) cells, human embryonic carcinomas cells (hEC) and adult stem cells including, but not limited to bone marrow cells. Other genetically modified cells that give rise to any cell type in the heart, including cardiomyocyte and vascular endothelial cells can also be used for the coating. Cultured immortalized cells including but not limited to Satellite cells and myoblasts can also be used for the coating. Stem cells and other cells can be modified by gene transfer using means well known in the art, for example chemical transfection, biological transfection or viral infection. Cell populations are purified using a variety of well known techniques including fluorescence-activating cell sorting (FACS) or magnetic-activated cell sorting (MACS), and resistant gene selections. (See generally, Robinson J. P. Handbook of Flow Cytometry Methods, Wiley-Liss, New York, 1993; Shapiro H. Practical Flow Cytometry, Third Edition, Alan R. Liss, New York, N.Y., 1994.)

The meshed tube 10 is put into a bioreactor together with stem cells in a culture medium containing all the nutrients including growth factors and small molecules. In order to facilitate cell growth, differentiation and migration on the meshed tubes 10, biological and nonbiological materials can be coated onto the meshed tubes during the culturing process. These approaches include but are not limited to use of growth factors, such as VEGF, Insulin growth factor, enzymes and small molecules. Also genetic materials such as cDNA for growth factors, transcriptional factors (e.g., myogenic factors or homeodomains) and cytokines are used during the culture process. In addition to growth factors and genetic material useful for cell growth, differentiation and migration, molecules such as rapamycin or FK560 are added to the matrices to help avoid immunorejection. As a result of the culturing process, the meshed tubes will contain millions of cells. The meshed tubes 10, once coated with stem cell formula 16, are then implanted into the scarred and/or damaged tissue region of the heart during surgical or via percutaneous catheter-based implantation. This device could also implant into other organs such as spinal cord, brain, kidney, liver.

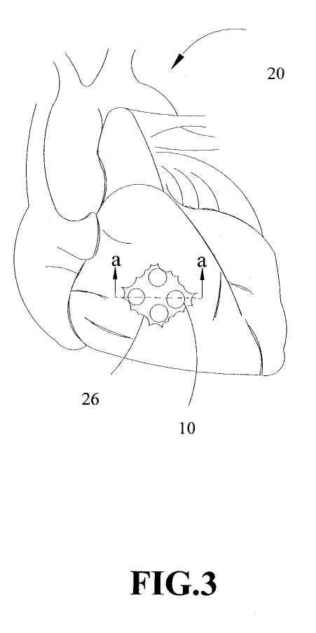

FIG. 3 is a top view of the embodiment of FIG. 1 placed into failing heart muscle area 26 of heart 20. Preferably, one or more devices 10 are placed into the failing heart muscle 26. Device 10 may be mechanically forced, or otherwise placed inside the failing heart muscle area 26 using surgical or percutaneous catheter- based implantation, and thereafter secured to the desired area. Device 10, when placed into an organ or tissue, will readily reach the necessary depth and breadth of damaged area thereby providing fully compatible cell repopulation of said damaged area.

Although the embodiment of the present invention illustrated in FIG. 3 shows the device 10 is inserted by force into the failing heart muscle area 26, it is understood by those of ordinary skill in the art that other embodiments for placing device 10 inside the failing heart muscle area 26 fall well within the spirit of the current invention. In one exemplary embodiment, placement channel(s) may be surgically created in the failing heart muscle area 26, in order to place device 10 inside. Device 10 thereafter may be sutured with the surrounding tissues. In a further embodiment, device 10 comprises an adhesive allowing the device to adhere to the failing heart muscle area 26. In a still further embodiment, mechanical insertion, surgical attachment and adhesive attachment are used in a combined approach to secure device 10 inside the failing heart area 26.

Although the embodiment of the present invention illustrated in FIG. 3 shows the device 10 is vertically inserted into the failing heart muscle area 26 from the top of the heart 20, it is to be understood that other embodiments exist in which device 10 may be placed inside the failing heart muscle area 26 with different orientation. In one exemplary embodiment, the device 10 may be inserted inside the failing heart muscle area 26 from the side of the heart 20. In other exemplary embodiment, the device 10 may be horizontally placed into the failing heart muscle area 26 from the top of the heart 20. The purpose of device 10 placement arrangement is to facilitate stem cell repopulation of heart muscles inside the heart failing area 26, thereby restoring the heart muscle pumping function.

Although the embodiment of the present invention illustrated in FIG. 3 shows that device 10 may be implanted in the failing heart muscle area 26 without any surgical operation on such area, it is understood that other embodiments exist in which the failing heart muscle area 26 is surgically crafted prior to the device 10 implant. In one such embodiment, striates of muscles are removed from the failing heart muscle area 26. Device 10 is then inserted via surgery or percutaneous catheter into the empty spaces resulting from the removal of the failing muscle. The purpose of crafting the failing heart muscle area 26 is to facilitate the stem cells to repopulate inside the area 26, to restore the heart muscle pumping function.

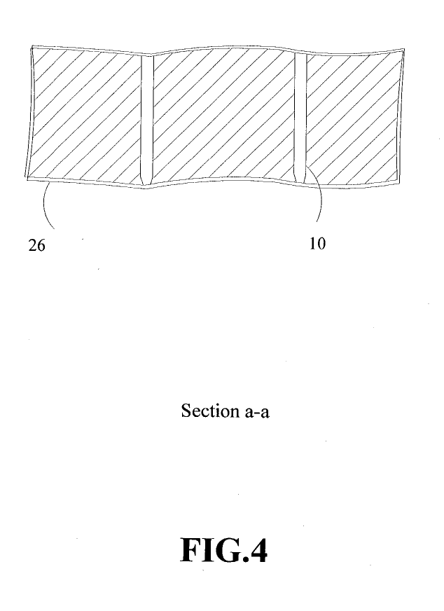

FIG. 4 is a cross section view and a transverse plane view of the embodiment of FIG. 3. The stem cell formula 16 coated on the device 10 delivers cells to the surrounding heart muscle area 26, thereby repopulating failing heart muscle area 26 with healthy cells, and thus restoring heart muscle function. Device 10 is preferably inserted into the heart muscle area 26 via percutaneous catheter; however, other methods such as surgical implant may also be used.

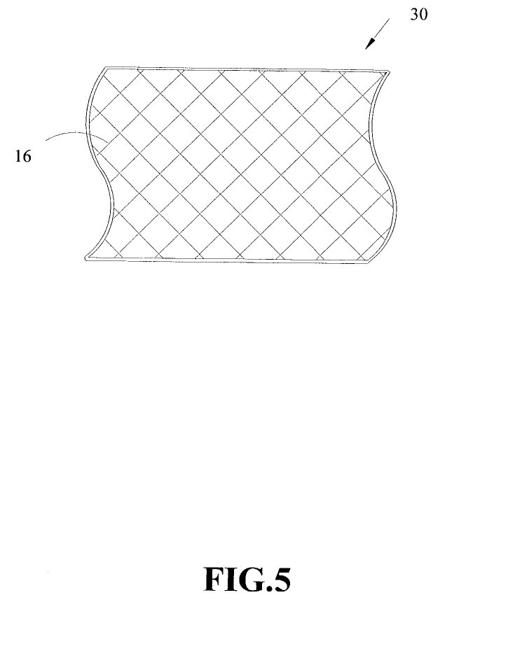

FIG. 5 illustrates a general example of meshed pad, useful for surgical or percutaneous catheter mediated in vivo placement according to one embodiment of the present invention. In one embodiment, the meshed pad 30 is coated with stem cell formula 16, thereby resulting in a cell coated device. The device 30 may be coated with formula 16 on one or both sides as described and referenced herein above. The method of how to coat stem cell onto the meshed pad is similar to how to coat the meshed tube with stem cell, also described herein above.

Although the embodiment of the present invention illustrated in FIG. 5 shows the device 30 is rectangle shaped pad with uniform thickness, it is obvious to those of ordinary skill in the art that the device may be shaped in any of a variety of configurations. In one embodiment, the device 30 may have different shape such as square, round, diamond, etc. In an additional embodiment, the device 30 is shaped to maximally deliver new cells to the damaged tissues. In a further embodiment, device 30 may have various thickness profiles along the length, as determined by the damaged tissues to be replaced. Those of ordinary skill in the art will readily shape device 30 of the current invention to meet a variety of transplant needs.

Preferably, the scaffolding matrix of FIG. 5 is a biocompatible material and/or a biodegradable or bioerodable such as those described in U.S. Pat. No.: 6,537,567. In a preferred embodiment of the present invention illustrated of FIG. 5, the meshed pad 30 may be fabricated from 304V stainless steel. Alternatively, other biocompatible metal materials such as nitinol may be used. In one exemplary embodiment, the present invention may be fabricated from bioabsorbable materials such as poly lactic acid (PLA), polyglycolic acid (PGA), polysebacic acid (PSA), poly(lactic-co-glycolic) acid copolymer (PLGA), poly(lactic-co-sebacic) acid copolymer (PLSA), poly(glycolic-co-sebacic) acid copolymer (PGSA), polyesters, polyorthoesters, polyanhydrides, polyiminocarbonates, inorganic calcium phosphate, aliphatic polycarbonates, polyphosphazenes, collagen based adhesive, fibrin based adhesive, albumin based adhesive, polymers or copolymers of caprolactones, amides, amino acids, acetals, cyanoacrylates, degradable urethanes; or biocompatible but non-bioabsorable materials such as acrylates, ethylene-vinyl acetates, non-degradable urethanes, styrenes, vinyl chlorides, vinyl fluorides, TEFLON® (DuPont, Wilmington, Del.), nylon, HYTREL (DuPont) or PEBAX (Autofina). The above disclosure is not an exhaustive list, but instead represents alternate embodiments illustrated by way of example only. Those of ordinary skill in the art are knowledgeable of and will readily employ the numerous biocompatible, biodegradable and bioerodable materials in the art in order to achieve the spirit of the current invention.

Although the embodiment of the present invention illustrated in FIG. 5 may have meshed design cuts, other pattern designs may be used. In one embodiment, the device 30 may have parallel cut lines spaced sufficiently apart to enable stem cells to repopulate inside the failing heart muscle area 26. The purpose of specific pattern design is to facilitate the stem cells growth depth and orientation inside the failing heart muscle area, to restore the heart muscle pumping function.

In one embodiment, device 30 may have a specific cut pattern design going through the wall thickness. It is to be understood that device 30 may have specific cut pattern designs on both sides, but the cuts may not go through the wall thickness.

Alternatively, in one embodiment, device 30 may not have patterned cuts. In this instance, device 30 may have smooth or rough surface on one side or both sides.

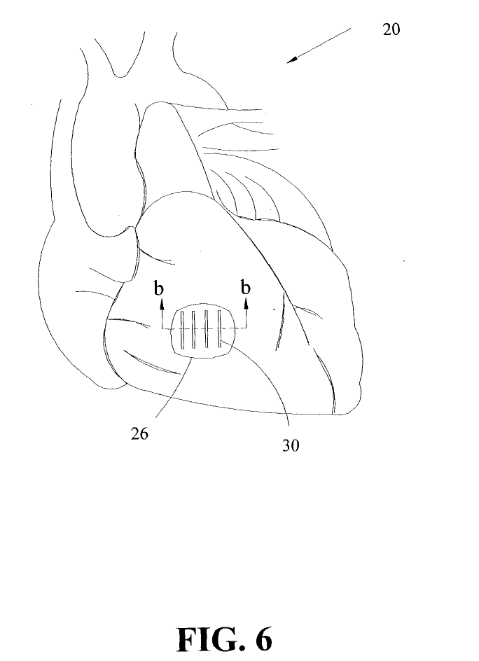

FIG. 6 is a top view of the embodiment of FIG. 5 placed into failing heart muscle area 26 of heart 20, via a surgical or a percutaneous catheter mediated technique. One or more devices 30 may be placed in area 26. Device 30 may be mechanically forced through heart muscle area 26, such as when a percutaneous catheter is the preferred delivery route. Alternatively, during surgery device 30 may be placed inside the failing heart muscle area 26 and thereafter secured.

Although the embodiment of the present invention illustrated in FIG. 6 shows the device 30 is inserted by force into the failing heart muscle area 26, it is understood by those of ordinary skill in the art that other embodiments for placing device 30 inside the failing heart muscle area 26 fall well within the spirit of the current invention. In one exemplary embodiment, placement channel(s) may be surgically created in the failing heart muscle area 26, in order to place device 30 inside said channels. Device 30 thereafter is sutured to the surrounding tissues. In a further embodiment, device 30 comprises an adhesive allowing the device to adhere to the failing heart muscle area 26. In a still further embodiment, mechanical insertion, surgical attachment and adhesive attachment are used in a combined approach to secure device 30 inside the failing heart area 26.

Although the embodiment of the present invention illustrated in FIG. 6 shows the device 30 is inserted into the failing heart muscle area 26 from the top of the heart 20, it is understood that other embodiments exist in which device 30 may be placed inside the failing heart muscle area 26 with different placement arrangements. In one exemplary embodiment, the device 30 may be placed inside the failing heart muscle area 26 from the side of the heart 20. The purpose of device 30 placement arrangements is to facilitate stem cells repopulation inside the heart failing area 26, to restore the heart muscle pumping function.

Although the embodiment of the present invention illustrated in FIG. 6 shows that device 30 may be implanted in the failing heart muscle area 26 without any surgical operation on such area by way of the percutaneous catheter, it is understood that other embodiments exist in which the failing heart muscle area 26 is surgically crafted prior to device 30 implant. In one embodiment, filaments of muscles are removed from the failing heart muscle area 26. Device 30 is then inserted inside the empty spaces resulting from the removal of the failing muscle filaments. The purpose of crafting the failing heart muscle area 26 is to facilitate the stem cells to repopulate inside the area 26, to restore the heart muscle pumping function.

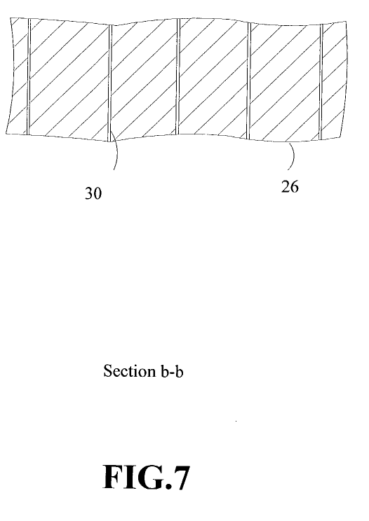

FIG. 7 is a cross section view and a transverse plane view of the embodiment of FIG. 6. The stem cell formula 16 is coated on the device 30 delivering cells to the surrounding heart muscle area 26, thereby repopulating failing heart muscle area 26 with healthy cells to restore heart muscle function. Device 30 is preferably inserted into the heart muscle area 26 via percutaneous catheter; however, other methods such as surgical implant may also be used.

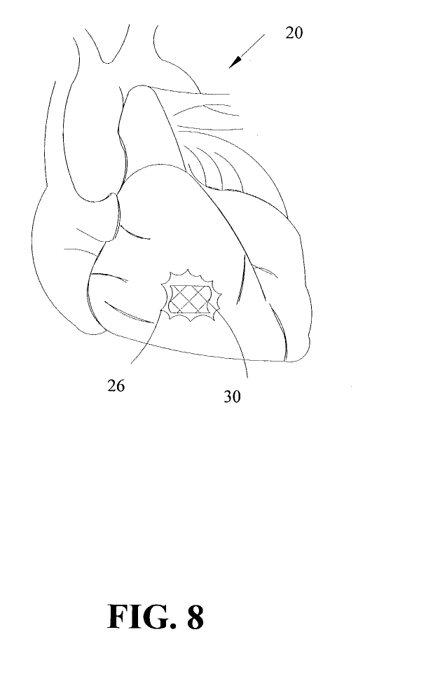

FIG. 8 is a top view of the embodiment of FIG. 5 attached to the surface of the failing heart muscle area 26 of heart 20 via surgical or percutaneous catheter mediated technique.

Although the embodiment of the present invention illustrated in FIG. 8 shows only one piece of device 30 attached to the surface of the failing heart area 26, it is understood that other embodiments exist in which multiple devices 30 are attached to the surface of the failing heart area 26 with different arrangements.

Although the embodiment of the present invention illustrated in FIG. 8 shows the device 30 adhered to the surface of the failing heart area 26 with its own adhesive, it is understood that other embodiments exist in which device 30 is attached to the surface of the failing heart area 26 through a different means. In one embodiment, device 30 is placed on the surface of the failing heart muscle area 26 and secured to such surface of the surrounding tissues using a surgical suture. In another embodiment, a surgical suture and an adhesive may be used as a combined approach to attach device 30 to the surface of the failing heart area 26.

Although the embodiment of the present invention illustrated in FIG. 8 shows that device 30 may be simply attached to the surface of the failing heart muscle area 26 without any surgical operation on such area, it is understood that other embodiments exist in which the surface of failing heart muscle area 26 is surgically crafted. In one embodiment, a layer of the failing heart muscle area 26 is removed from the top surface of heart 20. Device 30 is placed on heart 20 such that it fills the cavity created by the layer removal. In another embodiment, grooves are surgically created on the surface of the failing heart area 26 and device 30 is attached to such crafted surface thereafter.

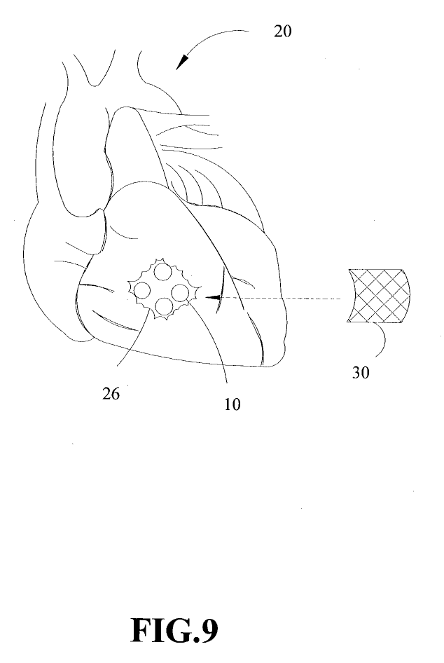

FIG. 9 is a top view of the embodiment of FIG. 1 implanted into the failing heart muscle area 26, and the embodiment of FIG. 5 attached to the surface of the area 26. In one embodiment, device 10 is mechanically inserted inside the failing heart muscle area 26 via surgical or percutaneous catheter of the current invention, and then device 30 is placed over the tops of the inserted device 10 and the remaining surface of area 26 with its own adhesive.

Although the embodiment of the present invention illustrated in FIG. 9 shows the device 10 is inserted by force into the failing heart muscle area 26, it is understood that other embodiments exist in which device 10 is attached to the failing heart muscle area 26 through a different means of attachment. In one exemplary embodiment, placement cuts are surgically created in the failing heart muscle area 26, in order to easily place device 10 inside. Device 10 thereafter is sutured with the surrounding tissues. Alternatively, in one embodiment, device 10 comprises an adhesive facilitating adhesion of device 10 to the failing heart muscle area 26.

Although the embodiment of the present invention illustrated in FIG. 9 shows the device 30 may adhere to the surface of the failing heart area 26 with its own adhesive, it is understood that other embodiments exist in which device 30 is attached to the failing heart muscle area 26 through a different means of attachment. In one embodiment, device 30 may be placed on the surface of the failing heart muscle area 26 and surgically sutured to the surrounding heart tissue. In another embodiment, surgical suture and an adhesive are used as a combination approach to attach device 30 to the surface of the failing heart area 26.

Although the embodiment of the present invention illustrated in FIG. 9 shows that device 10 is vertically placed inside the failing heart muscle area 26 from the top of the heart 20, and device 30 covers the surface of area 26 and device 10, it is to be understood that other embodiments exist in which device 10 and device 30 placed into failing heart muscle 26 in a different arrangement. In one exemplary embodiment, device 10 is inserted inside the failing heart muscle area 26 from the side of the heart 20, and device 30 covers the surface of area 26 and device 10. In one other embodiment, both device 10 and device 30 are inserted inside the failing heart area 26, and another device 30 covers the surface of area 26 and devices implanted inside area 26. The purpose of device 10 and device 30 combination placement arrangements is to facilitate stem cell repopulation of heart muscles inside the heart failing area 26, thereby restoring the heart muscle pumping function.

Although the embodiment of the present invention illustrated in FIG. 9 shows that device 10 and device 30 may be implanted in the failing heart muscle area 26 without any surgical operation on such area, it is understood that other embodiments exist in which the failing heart muscle area 26 is surgically crafted prior to device 10 and device 30 implant. In one embodiment, a layer of the failing heart muscle area 26 is removed from the top surface; device 10 is then inserted inside the failing heart muscle area 26, thereby filling in the empty space resulting from said layer removal. In another embodiment, striates of failing heart muscle are surgically removed from the area 26 and device 10 is thereafter placed into the spaces resulting from said surgical removal. Device 30 is then used to cover the surface of area 26 and the embedded device 10. In a further embodiment, striates of failing heart muscle are surgically removed from the area 26 and a layer of the failing heart muscle is removed from the top of area 26. Device 10 and device 30 are placed such that they fill in these empty spaces respectively. The purpose of crafting the failing heart muscle area 26 is to facilitate stem cells repopulation inside the area 26, thereby restoring the heart muscle pumping function.

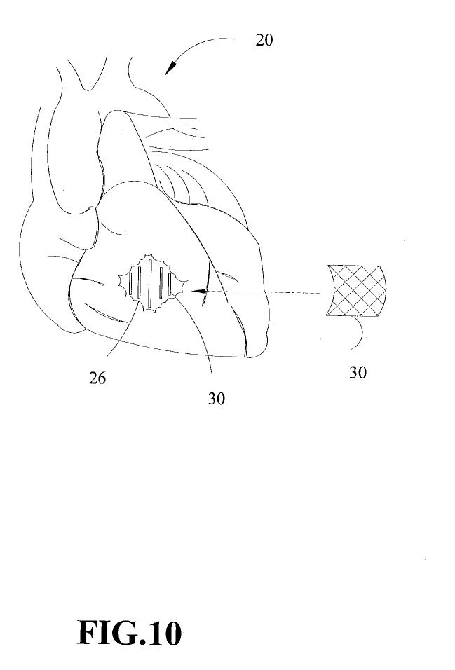

FIG. 10 is a top view of the embodiment of FIG. 5 implanted into the failing heart muscle area 26 and the embodiment of FIG. 5 attached to the surface of the area 26. In one embodiment, a first device 30 is mechanically inserted inside the failing heart muscle area 26 using the surgical or percutaneous catheter of the current invention, and then another device 30 covers the tops of the inserted first device 30 and the remaining surface of area 26 with its own adhesive.

Although the embodiment of the present invention illustrated in FIG. 10 shows the device 30 is inserted by force into the failing heart muscle area 26, it is understood that other embodiments exist in which device 30 is placed inside the failing heart muscle area 26 through a different means of attachment. In one exemplary embodiment, placement channel(s) are surgically created in the failing heart muscle area 26, in order to easily place device 30 inside the area 26, and device 30 is thereafter sutured onto the surrounding tissues. Alternatively, in one embodiment, device 30 comprises an adhesive and thus is adhered to the failing heart muscle area 26. In this instance, device 30 is not sutured onto the surrounding heart tissues.

Although the embodiment of the present invention illustrated in FIG. 10 shows that device 30 may adhere to the surface of the failing heart area 26 with its own adhesive, it is understood that other embodiments exist in which device 30 is attached to the surface of the failing heart area 26 by a different means. In one embodiment, device 30 is placed onto the surface of the failing heart muscle area 26 by surgically suturing it to the surrounding heart tissues. In another embodiment, a surgical suture and an adhesive are used in combination to attach device 30 to the surface of the failing heart area 26.

Although the embodiment of the present invention illustrated in FIG. 10 shows that a first device 30 is placed vertically inside the failing heart muscle area 26 from the top of the heart 20, and another device 30 covers the edges of the inserted first device 30 and the remaining surface of area 26, it is understood that other embodiments exist in which device 30 is attached to heart muscle area 26 via a different means. In one exemplary embodiment, one or more first device 30 is/are placed horizontally inside the failing heart muscle area 26 from the side of the heart 20, and another device 30 covers the surface of the failing heart area 26 and the first device 30 implanted inside the area 26. The purpose of device 30 combination placement arrangements is to facilitate stem cell repopulation inside the heart failing area 26, thereby restoring the heart muscle pumping function.

Although the embodiment of the present invention illustrated in FIG. 10 shows that device 30 may be implanted in the failing heart muscle area 26 without any surgical operation on such area, it is understood that other embodiments exist in which the failing heart muscle area 26 is surgically crafted prior to device 30 implant. In one embodiment, a layer of the failing heart muscle area 26 is removed from the surface and a first device 30 is inserted inside the failing heart muscle area 26, then another device 30 fills in the empty space resulting from said layer removal. In another embodiment, filaments of muscles are removed from the failing heart muscle area 26 and a first device 30 is inserted inside the empty spaces resulting from said removal. A second device 30 is then applied to heart 20 covering the surface of the failing heart muscle area 26 and said first device 30 implanted inside area 26. In a further embodiment, filaments of muscles are removed from the failing heart muscle area 26, and a layer of the failing heart muscle 26 is removed from top of area 26. First and second devices 30 are inserted into these empty spaces respectively. The purpose of crafting the failing heart muscle area 26 is to facilitate stem cells repopulation inside the failing heart muscle area 26, thereby restoring the heart muscle pumping function.

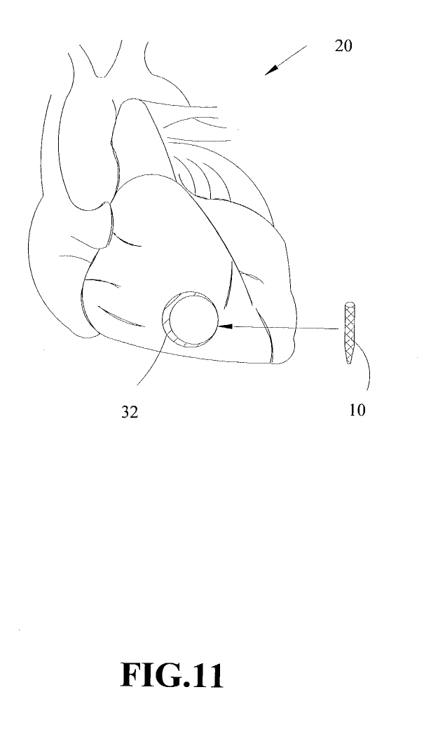

FIG. 11 is a perspective view of the embodiment of FIG. 1 implanted into a surgical site 32. In this instance, the surgical site 32 is where abnormal heart muscles, (i.e., failing, enlarged, and/or stiffened heart muscles), are surgically removed, thereby leaving a void.

In one embodiment, device 10 is sutured to the surrounding heart tissues of site 32. Alternatively, a plurality of devices 10 comprise an adhesive means and are adhered to the surgical site 32 and to each other. In this instance, device 10 is not sutured onto the surrounding heart tissues. In a further embodiment, surgical attachment and adhesive attachment are used in combination to secure device 10 to the surgical site 32.

In one embodiment, a single device 10 is inserted into the surgical site 32 of heart 20. In another embodiment, a plurality of devices 10 are inserted into the surgical site 32 of heart 20. Numerous factors play into a decision regarding the number of devices to use in the current invention, including but not limited to the magnitude of the surgical site 32.

In one embodiment, device 10 is implanted vertically inside the surgical site 32; however, those of ordinary skill in the art will readily place device 10 into the surgical site 32 with a variety of placement orientations, e.g. horizontally. The purpose of device 10 placement arrangement is to facilitate stem cell repopulation of heart muscles, thereby restoring the heart muscle pumping function.

Although the embodiment of the present invention illustrated in FIG. 11 shows only device 10 implanted inside the surgical site 32, it is understood that other embodiments exist in which device 30 may be implanted together with device 10. For example, device 30 may enclose the surgical site 32 after device 10 is implanted inside site 32. Alternatively, both device 10 and device 30 may be implanted inside the surgical site 32, and device 30 may enclose the surgical site 32. Numerous other configurations exist and are obvious to those of ordinary skill in the art given the current disclosure.

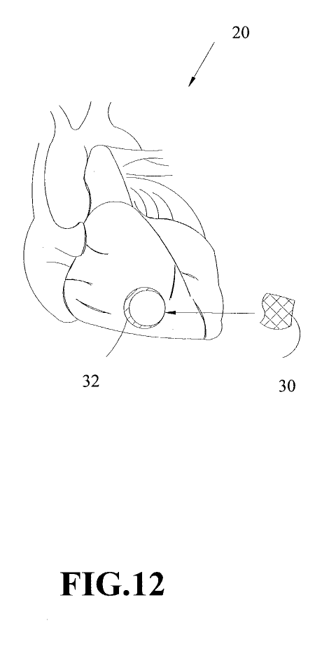

FIG. 12 is a perspective view of the embodiment of FIG. 5 implanted into a surgical site 32. In this instance, the surgical site 32 is where abnormal heart muscles, (i.e., failing, enlarged, and/or stiffened heart muscles), are surgically removed, thereby leaving a void.

In one embodiment, device 30 is sutured to the surrounding heart tissues of site 32. Alternatively, device 30 comprises an adhesive and thus is adhered to the surgical site 32, and, if a plurality of devices 30 are used, adhered to each other. In this instance, device 30 is not sutured onto the surrounding heart tissues. In a further embodiment, surgical attachment and adhesive attachment are used in combination to secure device 30 to the surgical site 32.

In one embodiment, there only one device 30 is inserted into the surgical site 32 of heart 20. In another embodiment, a plurality of devices 30 are inserted into the surgical site 32 of heart 20. Numerous factors play into a decision regarding the number of devices to use in the current invention, including but not limited to the magnitude of the surgical site 32.

In one embodiment, device 30 is implanted vertically inside the surgical site 32; however, those of ordinary skill in the art will readily place device 30 into the surgical site 32 with a variety of placement orientations, e.g. horizontally. In a further embodiment, a first device 30 is implanted inside the surgical site 32, and another device 30 may enclose the surgical site 32. The purpose of device 30 placement arrangements is to facilitate stem cells to repopulate heart muscles, to restore heart muscle pumping function. The purpose of device 30 placement arrangement is to facilitate stem cell repopulation of heart muscles, thereby restoring the heart muscle pumping function.

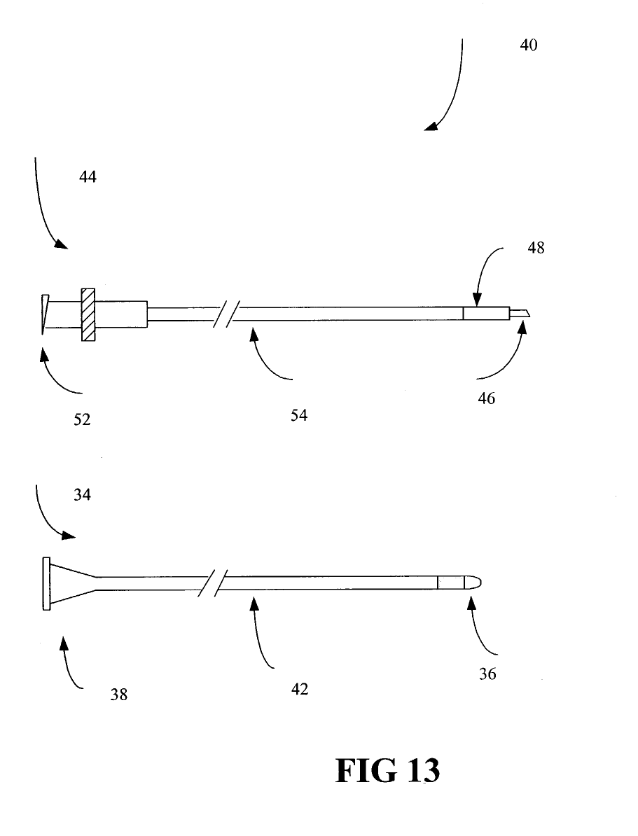

FIG. 13 illustrates a general example of percutaneous catheter-based implantation system 40 used to deliver the stem-cell coated matrices to failing heart muscle area 26 in the current invention.

In one embodiment, catheter system 40 comprises guiding catheter 34 and device delivery catheter 44. In this instance, guiding catheter 34 tracks to the targeted failing heart area 26 providing a guide path for device delivery catheter 44, and thereafter to failing heart area 26. In another embodiment, device delivery catheter 44 tracks to the targeted failing heart area 26 without guiding catheter 34.

Guiding catheter 34 has catheter body 42 with an open lumen to provide traveling path for device delivery catheter 44. Guiding catheter 34 has open hub 38 at one end to provide entrance for device delivery catheter 44. Distal tip 36 is to facilitate guiding catheter 34 to track smoothly to failing heart area 26. Guiding catheter 34 may be fabricated from biocompatible metal materials such as 304 V stainless steel, nitinol, or biocompatible polymer materials such as TEFLON® (DuPont, Wilmington, Del.), nylon, PEBAX® (Atofina), HYTREL®(DuPont), acrylates, ethylene-vinyl acetates, urethanes, styrenes, vinyl chlorides, vinyl fluorides. In one embodiment, the present invention may be fabricated from both metal materials and polymer materials. Guiding catheter 34 may be coated with silicone or a hydrophilic coating for better trackability. Guiding catheter 34 may have sections comprising radiopaque compounds to provide visibility under x-ray.

Device delivery catheter 44 has distal tip 46, body 54 and proximal end 52. Proximal end 52 of device delivery catheter 44 has connection mechanism for attaching accessories. For example, in one embodiment, proximal end 52 has a luer-lock function connected to an inflation/deflation indeflator. Device delivery catheter 44 may be fabricated from biocompatible metal materials such as 304 V stainless steel, nitinol, or biocompatible polymer materials such as TEFLON® (DuPont, Wilmington, Del.), nylon, PEBAX® (Atofina), HYTREL®(DuPont), acrylates, ethylene-vinyl acetates, urethanes, styrene, vinyl chlorides, vinyl fluorides. In one embodiment, device delivery catheter 44 may be fabricated from both metal materials and polymer materials. Device delivery catheter 44 may be coated with silicone or a hydrophilic coating for better trackability. Device delivery catheter 44 may have sections comprising radiopaque compounds to provide visibility under x-ray.

In one embodiment, the cell coated device is releasably secured onto section 48 of device delivery catheter 44. Distal tip 46 may be fabricated from a biocompatible metal material and shaped sharp enough to easily insert into failing heart muscle area 26, thereby guiding the present invention into failing heart muscle area 26. Once the cell coated device is mechanically forced inside failing heart muscle area 26, section 48 may be detached from the call coated matrix and thereafter withdrawn from failing heart muscle area 26. Placement channel(s) may be created in the failing heart muscle area 26 prior to the cell coated device placement.

In one embodiment, the cell coated device may be secured onto section 48 of device delivery catheter 44 by way of an adhesive. It is understood that other embodiments exist in which the cell coated device is secured onto section 48 of device delivery catheter 44 differently. By way of example only, the cell coated device may be hooked together with section 48 of device delivery catheter 44 using an adhesive.

In one embodiment, proximal end 52 of device delivery catheter 44 may be connected to a vacuum. Section 48 of device delivery catheter 44 will then collapse under negative pressure from a vacuum, thereby detaching from the present invention. It is to be understood that other embodiments exist for detaching the present invention from the device delivery catheter 44, including, but not limited to perforations in the material, rotational torque and/or longitudinal force. In one embodiment, a hook that secures the cell coated device together with section 48 may be removed or broken in order to detach the cell coated device from section 48.

Although the embodiment illustrated in FIG. 13 shows that the cell coated device is secured outside section 48 of device delivery catheter 44, it is understood that other embodiments exist for attaching the cell coated device to the device delivery catheter 44. In one embodiment, the cell coated device is attached to distal tip 46. The purpose of these types of arrangements is to achieve optimum implantation of the cell coated device into failing heart muscle area 26.

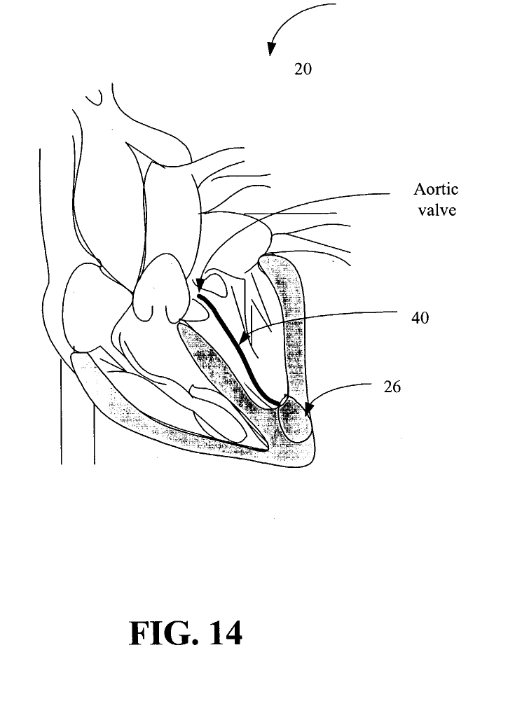

FIG. 14 illustrates a general example of catheter system 40 to deliver the cell coated device to failing heart muscle area 26 via a percutaneous catheter-based implantation. In one embodiment, catheter system 40 delivers the cell coated device into failing heart area 26. In another embodiment, catheter system 40 delivers the cell coated device to the surface of failing heart muscle area 26. In a further embodiment, catheter system 40 delivers the cell coated device to both the inside and the surface of failing heart muscle area 26.

Although the embodiment illustrated in FIG. 14 shows that catheter system 40 is inserted through aortic valve into left ventricle to reach failing heart muscle area 26, it is understood that other embodiments exist in which the cell coated device may be delivered to failing heart muscle area 26 differently, depending on the location of failing heart muscle area 26. In one exemplary embodiment, catheter system 40 is inserted through the tricuspid valve into the right ventricle.

Thus, the present invention as described herein provides several embodiments of a medical device for use in facilitating stem cell repopulation, repair and/or replacement of heart cells in an area of failing heart muscle, thereby restoring the heart's ability to pump blood. The medical device is made of biocompatible materials. The specific design of the device will facilitate the stem cells coated in the device to repopulate heart muscles inside the heart.

In the embodiments and examples presented herein, the present invention is described in relation to heart organ. It is to be understood that the present invention may be used other than heart. For example, the present invention may be used on liver, lung.

In the foregoing specification, the invention has been described with reference to specific exemplary embodiments thereof. It will, however, be evident that various modifications and changes may be made thereto without departing from the broader spirit and scope of the invention. These specifications and drawings are, accordingly, to be regarded in an illustrative rather than a restrictive sense.

What is claimed is:

1. A transplantable cell coated device comprising:
a structure for adhering to and growing cells; and
a transplant anchoring system.

2. The transplantable cell coated device of claim 1, wherein said device further comprises a releasable attachment mechanism.

3. The transplantable cell coated device of claim 1, wherein the structure and cells are compatible with the target location of a mammalian body.

4. The transplantable cell coated device of claim 1, wherein the structure and cells are compatible with the cardiac muscle of a human.

5. The transplantable cell coated device of claim 1, wherein the transplant anchoring system is compatible with the target location of a mammalian body.

6. The transplantable cell coated device of claim 5, wherein the transplant anchoring system is selected from the group consisting of adhesives, sutures, prongs, hooks and spikes.

7. The transplantable cell coated device of claim 5, wherein the cells are selected from the group consisting of stem-cells, human embryonic stem cells, human embryonic germ cells, human embryonic carcinomas cells, adult stem cells, and cardiomyocytes.

8. A method for delivering cells to a target location within a mammalian body;
comprising:
adhering to and growing cells on a compatible structure;
administering said cell coated structure to a tissue or organ;
anchoring said cell coated structure to said tissue or organ; and
allowing said cell coated structure to engraft into said tissue or organ.

9. The method of claim 8, wherein the cell coated structure is introduced via a surgical technique.

10. The method of claim 8, wherein the cell coated structure is introduced via a catheter based system.

11. The method of claim 10, further comprising the step of releasing said cell coated structure to said tissue or organ.

12. The method of claim 1 1, wherein the step for releasing the cell coated structure is selected from the group consisting of; mechanical force, rotational force, plunger, vacuum pressure and electrical separation.

13. The method of claim 8, wherein the cell coated structure is anchored to the tissue or organ by choosing from the group consisting of adhesives, sutures, hooks, prongs and spikes.

14. A delivery catheter comprising:
a catheter body comprising at least one lumen therethrough, and a flexible tubing having proximal and distal ends, wherein the proximal end is a connection mechanism;
a tip section comprising a first releasable attachment system;
a cell coated device comprising a second releasable attachment system and an implant securing mechanism, wherein the second releasable attachment system cooperates with the first releasable attachment system of the tip section.

15. The delivery catheter of claim 14, wherein the tip section is releasably connected to a cell coated device.

16. The delivery catheter of claim 15, wherein the tip section is released from the cell coated device using vacuum pressure.

17. The delivery catheter of claim 15, wherein the tip section is released from the cell coated device using mechanical force, rotational force, plunger, vacuum pressure or electrical separation.

18. The delivery catheter of claim 14, wherein the proximal end of the catheter body is connected to an accessory, and whereby the accessory is translocated through the lumen and to the distal end of the catheter body contacting the first releasable attachment system of the tip section.

19. The delivery catheter of claim 18, wherein the accessory is a vacuum, and whereby vacuum pressure is translocated through the lumen and to the distal end of the catheter body contacting the first releasable attachment system of the tip section.

20. The delivery system of claim 18, wherein the accessory is a plunger handle, and whereby the plunger shaft is translocated through the lumen and to the distal end of the catheter body contacting the first releasable attachment system of the tip section.

21. The delivery system of claim 18, wherein the accessory is a rotational torque device, and whereby the rotational torque device is translocated through the lumen and to the distal end of the catheter body contacting the first releasable attachment system of the tip section.

22. A method of treating damaged tissues and organs comprising:
adhering and growing cells on a biocompatible structure;
administering said cell coated structure to the damaged tissue or organ;
anchoring said cell coated structure to said tissue or organ at an optimal breadth and depth for repopulation of the damaged tissue or organ; and
allowing said cell coated structure to optimally engraft into said tissue or organ.

23. The method of claim 22, wherein the cell coated structure is administered via surgical techniques.

24. The method of claim 22, wherein the cell coated structure is administered via a catheter.

25. The method of claim 22, wherein the cell coated structure attached to the damaged tissue and/or organ by an anchoring step selected from the group consisting of adhesives, hooks, prongs, spikes and sutures.
